(19) United States
(12) Patent Application Publication
(10) Pub. No.: US 2008/0038198 A1
(43) Pub. Date: Feb. 14, 2008

(54) Method Of Screening Molecule Associated With Psychiatric Disorder

(75) Inventors: Ikue Mori, Aichi (JP); Yasushi Kajii, Tokyo (JP)

Correspondence Address: GREENBLUM & BERNSTEIN, P.L.C., 1950 ROLAND CLARKE PLACE, RESTON, VA, 20191, US

(73) Assignees: MITSUBISHI PHARMA CORPORATION, Osaka (JP); NATIONAL UNIVERSITY CORPORATION NAGOYA UNIVERSITY, Aichi (JP)

(21) Appl. No.: 10/596,985

(22) PCT Filed: Jan. 5, 2005
(86) PCT No.: PCT/JP05/00009

§ 371 Date: May 23, 2007

(30) Foreign Application Priority Data
Jan. 5, 2004 (JP) 000179/2004

(51) Int. Cl. A61K 49/00; A01K 67/00; A61P 43/00

(52) U.S. Cl. 424/9.2; 424/9.1; 800/9

(57) ABSTRACT

A method for screening a gene relating to a psychiatric disorder such as schizophrenia, which comprises the step of using a nematode behaviorally sensitized with amphetamine, methamphetamine, apomorphine, cocaine or the like for screening of a gene relating to the behavioral sensitization, and a method for screening a substance useful for prophylactic and/or therapeutic treatment of a psychiatric disorder, which comprises the step of using a nematode behaviorally sensitized with amphetamine, methamphetamine, apomorphine, cocaine or the like for screening of a substance that affects the behavioral sensitization.

### TECHNICAL FIELD

The present invention relates to a method for screening genes relating to psychiatric disorders such as schizophrenia.

### BACKGROUND ART

One of problems in the treatment of schizophrenia (also referred to as split personality) as a typical psychiatric disorder is poor prognosis. Prolonged latency of the disease and progressive increase in vulnerability for the onset are observed in many clinical cases (J. Abnorm. Psychol., 86, pp. 103-126, 1977; Br. J. Psychiatry, 155, pp. 15-21, 1989), and therefore, it is important to identify and analyze genes responsible for this disease.

Characteristics of recurrence of mental conditions in patients with chronic schizophrenia have also been reported in association with psychostimulant insanity, and a lot of similarities have been pointed out in these two diseases (Biol. Psychiatry, 18, pp. 429-440, 1983; Neuropsychopharmacology, 17, pp. 205-229, 1997; Brain Res. Rev., 31, pp. 371-384, 2000). Further, it has been reported that a common characteristic of progressive increases in vulnerability, by which abnormal behavior is induced in response to drugs and stress, is observed in both of the diseases (Brain Res., 514, pp. 22-26, 1990; Psychopharmacology, 151, pp. 99-120, 2000; Brain Res. Rev., 25, pp. 192-216, 1997). Accordingly, it is important to elucidate the relationship between these diseases at the genetic level for prophylactic and therapeutic treatment of these diseases.

In order to determine an association of a certain gene with a psychiatric disorder, for example, laboratory animals such as transgenic mice into which a specific gene is exogenously introduced and knockout mice in which a function of the gene is terminated have been conventionally used. A technique for determining whether a gene is affected by a dopamine agonist has been adopted, in which a dopamine agonist such as apomorphines and amphetamines is given to the laboratory animals, and behavior of the animals is examined by, for example, observing locomotion and examining changes in the number of times they cross a line and the like.

However, this technique has a problem in that considerable time and burden are required to make the transgenic mice or knockout mice. Therefore, development of a convenient and inexpensive method has been desired for determining whether a certain gene is associated with a psychiatric disorder.

For example, as for the laboratory animals used as a tool for the analyses of psychiatric disorders, laboratory animals behaviorally sensitized by giving a central nervous system stimulant or another dopamine agonist are reported to be useful for screening of therapeutic agents for psychiatric disorders such as schizophrenia, amphetamine psychosis, and drug dependence, or for analyses of disease-associated genes (Japanese Patent Application No. 2002-232448). Behavioral sensitization phenomena induced by central nervous system stimulants such as psychostimulants and cocaine or other dopamine agonists have been detailedly analyses in rodents (Brain Res., 514, pp. 22-26, 1990; Psychopharmacology, 151, pp. 99-120, 2000; Brain Res. Rev., 25, pp. 192-216, 1997). Further, cocaine-induced behavioral sensitization in drosophila, which is excellent for genetic analysis, was reported (Science, 285, pp. 1066-1068, 1999; Curr. Biol., 9, pp. R770-R772, 1999). However, these methods have a problem in that considerable burden and expenses are required, and thus an efficient and inexpensive method for gene analysis has been desired.

Nematodes (Caenorhabditis elegans) are multicellular organisms consisting of about 1000 cells that have a body length of about 1 mm and live in the soil. They are suitable for experiments to examine life duration because their life span is about 22 days at the longest. Although they are lower organisms, their basic structures of the body such as nerves, muscles, reproductive organs, and gastrointestinal tract are very similar to those of higher organisms such as humans. Further, the genome project of nematodes has been completed in 1998, and they are estimated to have about 19,000 genes.

Nematodes are known to utilize dopamine (DA) as a nerve transmitter substance and have a DA receptor similar to D1 and D2 in mammals (Neurosci. Lett., 319, pp. 13-16, 2002; J. Neurochem., 86, pp. 869-878, 2003) and DA transporter (DAT) (Mol. Pharmacol., 54, pp. 601-609, 1998; Proc. Natl. Acad. Sci. USA., 99, pp. 3264-3269, 2002; Annu. Rev. Pharmacol. Toxicol., 43, pp. 521-544, 2003). Since the primary site of action of the central nervous system stimulants, cocaine and amphetamines, is DAT, it is expected that these drugs effect nematodes via the DA system in the nerves. Further, it is considered that associative learning is established in nematodes in various ways, and that many of their mechanisms of neuroplasticity are similar to those of mammals (Behav. Brain. Res., 37, pp. 89-92, 1990; J. Neurobiol., 54, pp. 203-223, 2003). Therefore, it has been pointed out that, if behavioral sensitization induced by central nervous system stimulants or other dopamine agonists is established in nematodes, it may be a phenomenon based on the mechanism of neuroplasticity associated with changes in gene expression and morphological changes of synapses (J. Neurosci., 17, pp. 8491-8497, 1997; Eur. J. Neurosci., 11, pp. 1598-1604, 1999; Nat. Neurosci., 4, pp. 1217-1223, 2001; J. Neurochem., 85, pp. 14-22, 2003).

Further, by using nematodes, efficient isolation of a mutant deficient in a specific gene is achievable. Further, it is also possible to destroy specific nerve cells with laser beams, and analyze how the nerve cells associate with a phenomenon of interest. In recent years, it becomes possible to perform electrophysiological analysis with small nerve cells of nematodes, which was not achievable in the past, and the RNA interference method (henceforth also abbreviated as “RNAi”), in which gene expression is inhibited by using nematodes, was reported in 1998 (Nature, 391 (6669), pp. 806-11, 1998). Thus, convenient and inexpensive gene analysis methods using nematodes are known.

However, the genetic analysis using nematodes is performed primarily for analyses of genes associated with development, and no report has suggested or taught so far the use of nematodes for analysis of genes associated with psychiatric disorders.

### DISCLOSURE OF THE INVENTION

### Object to be Achieved by the Invention

An object of the present invention is to provide a method for efficiently and inexpensively screening a gene relating to a psychiatric disorder such as schizophrenia.

### Means for Achieving the Object

The inventors of the present invention conducted various researches to achieve the foregoing object. As a result, they found for the first time that nematodes were successfully sensitized behaviorally by central nervous system stimulants or other dopamine agonists. They also found that by using these behaviorally sensitized nematodes, genes relating to psychiatric disorders such as schizophrenia were successfully screened, and screening of a substance useful for prophylactic and/or therapeutic treatment of psychiatric disorders was achievable.

The present invention was accomplished on the basis of the aforementioned findings.

The present invention thus provides a method for screening a gene relating to a psychiatric disorder, which comprises the step of using a behaviorally sensitized nematode for screening a gene relating to the behavioral sensitization.

According to preferred embodiments, the present invention provides the aforementioned screening method, wherein the psychiatric disorder is schizophrenia; the aforementioned screening method, wherein a nematode behaviorally sensitized by a central nervous system stimulant or another dopamine agonist is used; the aforementioned screening method, wherein a nematode behaviorally sensitized by any of a psychostimulant, an apomorphine and a narcotic analgesic is used; and the aforementioned screening method, wherein a nematode behaviorally sensitized by any of amphetamine, methamphetamine, apomorphine and cocaine is used.

From another aspect, the present invention also provides a method for screening a substance useful for prophylactic and/or therapeutic treatment of a psychiatric disorder, which comprises the step of using a behaviorally sensitized nematode for screening a substance that effects the behavioral sensitization.

According to the preferred embodiments of the aforementioned screening method of the invention, there are provided the aforementioned screening method, wherein the psychiatric disorder is schizophrenia; the aforementioned screening method, wherein a nematode behaviorally sensitized by a central nervous system stimulant or another dopamine agonist is used; the aforementioned screening method, wherein a nematode behaviorally sensitized by any of a psychostimulant, an apomorphine and a narcotic analgesic is used; and the aforementioned screening method, wherein a nematode behaviorally sensitized by any of amphetamine, methamphetamine, apomorphine and cocaine is used. The present invention also provides a medicament comprising a substance selected by the aforementioned screening method as an active ingredient and used for prophylactic and/or therapeutic treatment of a psychiatric disorder.

From yet another aspect, the present invention also provides a behaviorally sensitized nematode, which is used for screening a gene relating to a psychiatric disorder or a substance useful for prophylactic and/or therapeutic treatment of a psychiatric disorder.

According to preferred embodiments of the aforementioned nematode, the present invention provides the aforementioned nematode, wherein the psychiatric disorder is schizophrenia; the aforementioned nematode, which is behaviorally sensitized by a central nervous system stimulant or another dopamine agonist; the aforementioned nematode, which is behaviorally sensitized by any of a psychostimulant, an apomorphine and a narcotic analgesic; and the aforementioned nematode, which is behaviorally sensitized by any of amphetamine, methamphetamine, apomorphine and cocaine.

### Effect of the Invention

The present invention provides a method for efficiently and inexpensively screening a gene relating to a psychiatric disorder such as schizophrenia.

### BRIEF DESCRIPTION OF THE DRAWINGS

FIG. 1 shows slowing responses and establishment of behavioral sensitization in nematodes in response to methamphetamine (henceforth also abbreviated as “MAP”). MAP in the assay plate inhibited locomotion of nematodes in a concentration-dependent manner. *p<0.05, **p<0.01 vs vehicle (Dunnett test, n=16).

FIG. 2 shows slowing responses and establishment of behavioral sensitization in nematodes in response to MAP. In the nematodes which experienced MAP on the previous day, increased susceptibility to a treatment with MAP was observed on the next day. ††p<0.001 vs vehicle-vehicle, ‡‡p<0.001 vs MAP-vehicle (Dunnett test, n=16-32), **p<0.0001 vs each vehicle-pretreated control (t-test, n=32).

FIG. 3 shows slowing responses to MAP and establishment of behavioral sensitization in nematodes. Establishment of behavioral sensitization was found to be dependent on the MAP concentration used for pretreatment. **p<0.01 vs vehicle (0 mM) (Dunnett test, n=16).

FIG. 4 shows long-term prolonged action of MAP behavioral sensitization in nematodes. Effect of the MAP washout period, i.e., time from a pretreatment to a challenge, on behavioral sensitization was analyzed. **p<0.01 vs each vehicle control (t-test, n=16).

FIG. 5 shows cross of behavioral sensitization. APO induced slowing responses in a concentration-dependent manner. *p<0.05, **p<0.01 vs vehicle (Dunnett test, n=16).

FIG. 6 shows cross of behavioral sensitization. An MAP pretreatment increased susceptibility to APO and induced slowing responses even at a low concentration. **p<0.01 (t-test, n=16).

FIG. 7 shows specificity of behavioral sensitization. IMI increased egg laying behavior in a concentration-dependent manner. The numerical values indicate the numbers of eggs counted for each 4 nematodes/plate.

FIG. 8 shows specificity of behavioral sensitization. An MAP pretreatment had no effect on IMI-induced egg-laying behavior.

FIG. 9 shows inhibition of establishment of MAP-induced behavioral sensitization in nematodes by an ondansetron treatment. **p<0.0001 vs vehicle-vehicle (t-test, n=14, 15), †p<0.05 vs MAP-vehicle (Dunnett test, n=13-16). In the drawing, OND (1) indicates the results of the treatment using 1 μM ondansetron; and OND (2) indicates the results of the treatment using 10 μM ondansetron.

### BEST MODE FOR CARRYING OUT THE INVENTION

When central nervous system stimulants, other dopamine agonists and the like are given to nematodes, which have outstanding characteristics as molecular neurobiological laboratory animals, behavioral sensitization characteristic to psychiatric disorders such as schizophrenia is established. The screening method of the present invention is a method for screening a gene relating to a psychiatric disorder or a method for screening a substance useful for prophylactic and/or therapeutic treatment of a psychiatric disorder, and is characterized by the use of nematodes behaviorally sensitized as described above.

Various information on nematodes (C. elegans) is provided by the Japanese Nematological Society. As for experimental methods using nematodes, detailed information on methods for morphological observation, culture of nematodes and the like can be obtained by referring to “The Nematode Caenorhabditis elegans” (Wood W. B., Cold Spring Harbor Laboratory Press). Therefore, those skilled in the art can easily obtain and utilize nematodes for the present invention.

The behavior to be sensitized in nematodes is not particularly limited. An example includes locomotion of nematodes or the like. However, the behavior is not limited to the above example, and may be any of behaviors that can be observed under microscope or the like, for example, pharynx pumping.

Types of agents used for behavioral sensitization in nematodes are not particularly limited. For example, nematodes can be behaviorally sensitized by using central nervous system stimulants or other dopamine agonists. More specifically, it is preferable to use nematodes behaviorally sensitized by any of a psychostimulant, an apomorphine, and a narcotic analgesic, and most preferably, nematodes behaviorally sensitized by any of amphetamine, methamphetamine, apomorphine and cocaine can be used. However, agents used for the behavioral sensitization are not limited to these examples.

According to the screening method of the-present invention, for example, a nematode with inhibited gene expression is constructed beforehand by a method for inhibiting expression of a specific gene, for example, by means of RNAi or the like, and the resulting nematode is treated with an agent that induces behavioral sensitization characteristic to a psychiatric disorder. Then, after a washout period, whether or not the gene associates with the establishment of behavioral sensitization characteristic to the psychiatric disorder can be judged based on the presence or absence of establishment of behavioral sensitization in response to a stress loaded with the agent. For the method for constructing a nematode with inhibited expression of a specific gene, for example, “The Nematode Caenorhabditis elegans” (Wood W. B., Cold Spring Harbor Laboratory Press) can be referred to. If behavioral sensitization is not established, the gene is determined to be a gene relating to the psychiatric disorder. In this case, a substance that inhibits expression of the gene is useful as an active ingredient of a medicament for prophylactic and/or therapeutic treatment of the psychiatric disorder. If behavioral sensitization is promoted, the gene is determined to be a gene relating to inhibition of onset of the psychiatric disorder. In this case, a compound that increases expression of the gene is useful as a therapeutic agent for the psychiatric disorder.

According to the present invention, the following two methods can be used as typical examples of the methods for screening target genes to be treated which relate to vulnerability to the onset of psychiatric disorders such as schizophrenia, manic depression, and drug dependence.

### (1) Screening by Forward Genetics

A mutation can be randomly introduced into the genome of C. elegans by applying a reported method (Dev. Biol., 221, pp. 295-307, 2000). An individual having the mutation in the gene relating to MAP-induced behavioral sensitization can be found by comparing MAP-induced behavioral sensitization in mutants with that in a wild type. According to a method of published report, the gene into which the mutation is introduced can be identified in the mutant found (Dev,Biol., 221, pp. 295-307, 2000).

### (2) Screening by Reverse Genetics

A knockout library of C. elegans in which deletions of a predetermined level are introduced over the entire genome of C. elegans is constructed according to a reported method, and the library can be screened by PCR for a mutant in which deletion occurs in a specific gene (Nat. Genet., 17, pp. 119-121, 1997). A gene relating to behavioral sensitization can be identified by analyzing MAP-induced behavioral sensitization by using animals in which the specific isolated and identified gene is knocked out and comparing the result with that obtained by using a wild type.

The screening method of another embodiment typically comprises at least (1) the step of introducing a test gene into a nematode, and (2) the step of measuring behavioral changes in the nematode due to a subsequent drug treatment or stress to the nematode. As for the method for introducing a gene into a nematode in an expressible state, for example, “The Nematode Caenorhabditis elegans” (Wood, W. B., Cold Spring Harbor Laboratory Press) can be referred to. If behavioral sensitization is not established, the gene can be determined to be a gene relating to inhibition of onset of a psychiatric disorder. In this case, a substance that promotes expression of the gene is useful as an active ingredient of a medicament for prophylactic and/or therapeutic treatment of the psychiatric disorder. If behavioral sensitization is promoted, the gene can be determined to be a gene relating to onset of a psychiatric disorder. In this case, a compound that inhibits expression of the gene is useful as a therapeutic agent of the psychiatric disorder. A novel or known arbitrary gene can be used as the gene.

Further, according to the screening method of the present invention, a nematode is treated beforehand with an agent that induces behavioral sensitization characteristic to a psychiatric disorder, then the treatment with the agent is discontinued and a treatment with a test substance is performed, followed by observation of whether behavioral sensitization is inhibited by subsequent stress load, for example, a treatment with amphetamine or the like. By means of the aforementioned procedure, it can be determined whether the test substance is useful as an active ingredient of a medicament for prophylactic and/or therapeutic treatment of the psychiatric disorder, more specifically, whether the substance has a pharmacological effect of regulating progression of behavioral sensitization characteristic to the psychiatric disorder. The type of the test substance is not particularly limited, and may be antibodies, antisense nucleic acids, RNAi and the like, as well as low molecular weight compounds, natural substances and the like.

### EXAMPLES

The present invention will be explained more specifically with reference to examples. However, the scope of the present invention is not limited to the following examples.

### Example 1

### Effect of Methamphetamine on Locomotion of Nematodes and Behavioral Sensitization

### (Experimental Method)

Breeding of Nematode:

Nematodes were bred on a lawn of E. coli OP50 coated on a 60-mm NGM plate (Genetics, 77, pp. 71-94, 1974) in an incubator at 20° C.

Drug Treatment and Behavior Observation:

MAP hydrochloride was dissolved in sterilized pure water at a 100-fold concentration, and 50 μL of the solution was applied to a 35-mm NGM plate (5 mL). As a control group, sterilized water used as a solvent was similarly applied to prepare an assay plate. Apomorphine (hereinafter also abbreviated as “APO”) dissolved in 0.1% aqueous ascorbic acid as a solvent was applied as a solution of 100-fold concentration in a similar manner to prepare an assay plate. For a control group, 0.1% aqueous ascorbic acid was used. The periphery of each assay plate was applied with a 3.5 mol/L sucrose solution to prevent nematodes from invading the back of the plate. Adult-stage nematodes bred on the E. coli lawn were washed with S-basal buffer (Genetics, 77, pp. 71-94, 1974), then transferred to each of the aforementioned assay plates, and left at room temperature with light shielding for one hour in the MAP treatment or 30 minutes in the APO treatment, and the number of body bends over 20 seconds was counted under a stereoscopic microscope. In the behavioral sensitization analysis, nematodes treated on the MAP-containing NGM plate for one hour were returned to the E. coli lawn, bred at 20° C. for a predetermined time and then treated with various drugs, and their behavior was observed.

Imipramine Treatment:

Nematodes washed with S-basal buffer were transferred onto a 35-mm assay plate prepared by applying imipramine (hereinafter abbreviated as “IMI”) in the same manner as that used for MAP, and the number of eggs laid during 90 minutes was counted. Five nematodes were transferred to each plate, and the count was obtained for each plate.

### Experiment 1

It is known that the DA signal plays a major role in real-time recognition of the presence or absence of bacteria as feed (Escherichia coli), as one kind of important environmental information. Specifically, locomotion on a plate without feed is more suppressed than that under a condition with feed, and nematodes behave to stay longer in that environment. This slowing response is regulated by the DA system in the nerves (Neuron, 26, pp. 619-631, 2000; J. Neurosci., 21, pp. 5871-5884, 2001). MAP functions as an indirect DA agonist in mammals by acting on DAT of presynapse to elevate the DA concentration between synapses (Eur. J. Pharmacol., 361, pp. 269-275, 1998), and accordingly, it was inferred that MAP induced the slowing responses in nematodes.

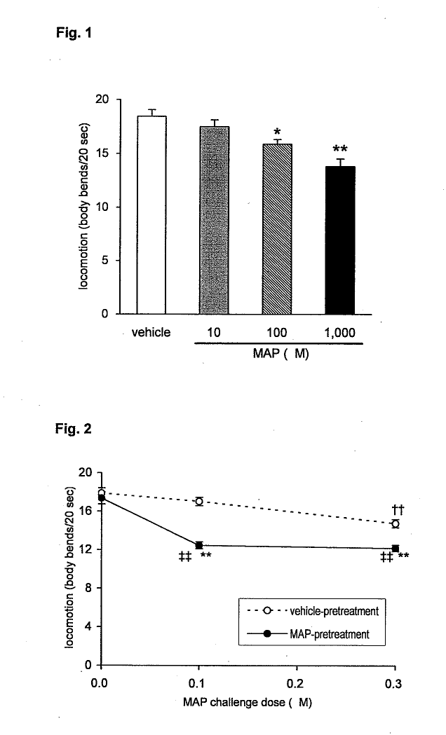

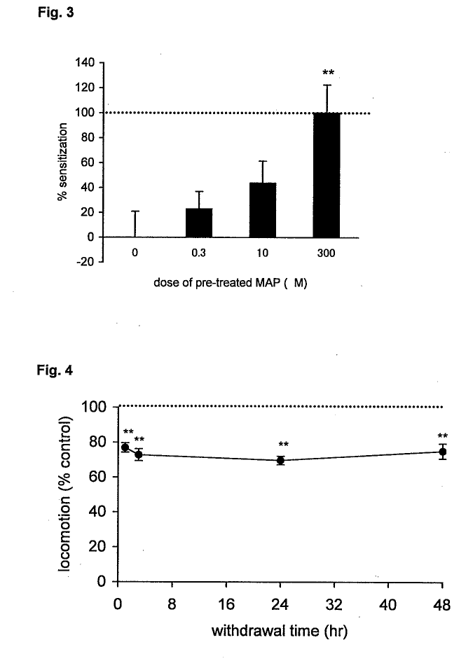

As shown in FIG. 1, MAP applied on a plate containing no feed inhibited the locomotion in a concentration-dependent manner and induced a slowing response mimicking the existence of feed. Further, effect of the presence or absence of MAP experience on this MAP-induced slowing response was analyzed, and as a result, it was revealed that the susceptibility was increased by experiencing 300 μmol/L of MAP on the previous day, leading to establishment of behavioral sensitization (FIG. 2). Establishment of MAP-inducted behavioral sensitization confirmed by using this slowing response as an index was dependent on the concentration of MAP experienced on the previous day, and a pretreatment with MAP at a concentration of a certain level or higher was necessary to statistically significantly establish the behavioral sensitization (FIG. 3).

From the above results, it was confirmed for the first time that MAP-induced behavioral sensitization showing characteristics common to that in mammals was also established in nematodes. Specifically, it was found that susceptibility to MAP or other DA agonists used in the subsequent treatment was continuously increased for a prolonged time by experiencing MAP beforehand.

### Example 2

### Characteristics of Methamphetamine-Induced Behavioral Sensitization in Nematodes

It is known that behavioral sensitization induced by central nervous system stimulants in mammals is a prolonged phenomenon associated with morphological changes in synapses (J. Neurosci., 17, pp. 8491-8497, 1997; Eur. J. Neurosci., 11, pp. 1598-1604, 1999; Nat. Neurosci., 4, pp. 1217-1223, 2001; J. Neurochem., 85, pp. 14-22, 2003). Under the experimental conditions in the present study, it was confirmed that the MAP-induced behavioral sensitization in nematodes was a phenomenon prolonged for at least 2 days (FIG. 4). Since the period for alteration of generations in nematodes is about 2 days (Curr. Biol., 4, pp. 151-153, 1994), the MAP-induced behavioral sensitization in nematodes found in the present study appears to be a semi-permanent change in nervous function that sustains over an extremely long period for nematodes.

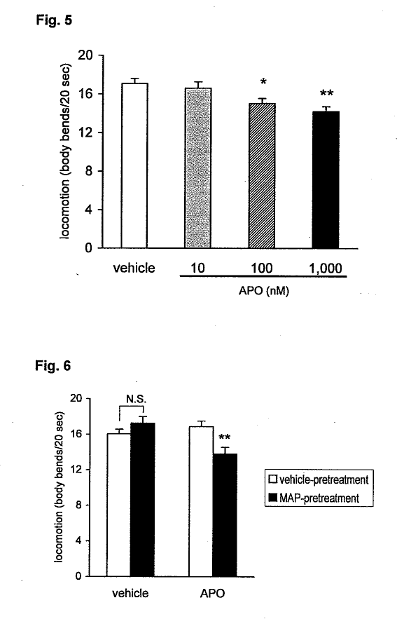

Another characteristic of the behavioral sensitization is that cross sensitization is observed over drugs that differ in the sites and modes of action. Accordingly, effect of a pretreatment with MAP, i.e., an indirect DA agonist that acts on DAT in presynapse, on susceptibility to APO which is a direct DA agonist that acts on DA receptor in postsynapse was analyzed. Acute treatment with APO inhibited the locomotion in a concentration-dependent manner as MAP and induced a slowing response (FIG. 5). Further, this APO-induced slowing response was enhanced by an MAP pretreatment, and cross of behavioral sensitization was observed (FIG. 6).

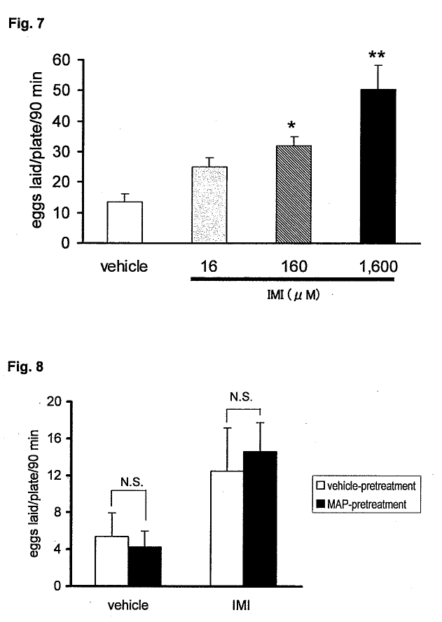

To examine a possibility that the observed MAP-APO cross behavioral sensitization depends on non-specific mechanisms such as change in the absorption system for the agent applied on a plate, analysis was performed to know whether susceptibility to IMI, which affects egg laying behavior primarily via the serotonin system, was effected by an MAP pretreatment. It was confirmed that IMI applied on an NGM plate promoted egg laying in a concentration-dependent manner (FIG. 7) as reported previously (J. Neurosci., 15, pp. 6975-685, 1995). Subsequently, influence of the MAP pretreatment on this effect of IMI was examined. As a result, the MAP treatment on the previous day had no influence on the egg-laying behavior promoted by IMI (FIG. 8). Therefore, certain specificity in cross of behavioral sensitization induced by MAP and APO was confirmed.

The experimental results suggested that the phenomena of cross sensitization between indirect and direct DA agonists and sustainability thereof are an aspect of the neuroplasticity as in mammals, and it was inferred that at least a part of the mechanism is common to that in mammals.

### Example 3

### Screening for Chemical Substance Inhibiting Establishment of Behavioral Sensitization in Nematodes

### (Experimental Method)

Breeding of Nematode:

In an incubator inside of which was maintained at 20° C., nematodes were bred on an Escherichia coli lawn prepared by applying the E. coli OP50 strain on a 60-mm NGM plate (Genetics, 77, pp. 71-94, 1974).

Drug Treatment and Behavior Observation:

MAP hydrochloride was dissolved in pure water to prepare a 30 mM solution, and 50 μL of the solution was applied to a 35-mm NGM plate (5 mL) to prepare a 300 μM MAP plate. This MAP plate was further applied with 50 μL of an ondansetron solution (0.1 or 1 mM) dissolved in 100 mM NaCl or 0.1 N hydrochloric acid and adjusted to neutral pH by using NaOH to complete an assay plate. Nematodes (adults) bred on the Escherichia coli lawn were washed with S-basal buffer (Genetics, 77, pp. 71-94, 1974) for a short period of time, and then incubated on each of the aforementioned assay plates at 20° C. for one hour with light shielding. For this procedure, the periphery of each assay plate was applied with a 3.5 M sucrose solution to prevent nematodes from invading the back of the assay plate. After completion of the incubation for one hour, the nematodes were transferred to a plate with an Escherichia coli lawn prepared on a 35-mm NGM plate (5 mL) coated with 50 μL of 100 mM NaCl or a neutral ondansetron solution (0.1 or 1 mM), and incubated overnight at 20° C. with light shielding. On the following day, the nematodes were washed with S-basal buffer for a short period of time and transferred onto a MAP challenge plate prepared by applying 50 μL of a 10 μM MAP solution on a 35-mm NGM plate (5 mL), and the number of body bends over 20 seconds was counted under a stereoscopic microscope.

### (Results)

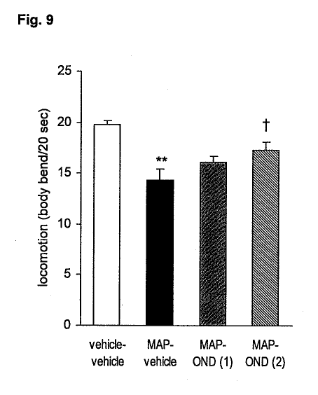

Pharmacological actions of ondansetron, widely used as an antiemetic drug, on the central nerve system have been reported, including a therapeutic effect for mental conditions (elicitation of hallucination and delusion), which develop during dopamine replacement therapy for Parkinson's disease (Neurology, 45, pp. 1305-1308, 1995; Scand. J. Rheumatol. Suppl., 113, pp. 37-45, 2000), effectiveness in initial treatment of alcoholism (Psychopharmacology, 149, pp. 327-344, 2000), and the like. However, its detailed action mechanism remains unknown. As shown in FIG. 9, ondansetron exhibits an antagonistic action on the establishment of MAP-induced behavioral sensitization in nematodes, and this result is consistent with that obtained with the evaluation system using rats described in Japanese Patent Application No. 2002-232448. Therefore, screening for the compound requiring one month in rats was completed only in 2 days by using the method of the present invention, and thus high usefulness of the method of the present invention was demonstrated.

### INDUSTRIAL APPLICABILITY

The present invention provides a method for efficiently and inexpensively screening a gene relating to a psychiatric disorder such as schizophrenia. The present invention also provides a method for screening a gene relating to a psychiatric disorder or a substance useful for prophylactic and/or therapeutic treatment of the psychiatric disorder.

1. A method for screening a gene relating to a psychiatric disorder, which comprises using a behaviorally sensitized nematode for screening of a gene relating to the behavioral sensitization.

2. A method for screening a substance useful for prophylactic and/or therapeutic treatment of a psychiatric disorder, which comprises using a behaviorally sensitized nematode for screening of a substance that affects the behavioral sensitization.

3. The screening method according to claim 1, wherein the psychiatric disorder is schizophrenia.

4. The screening method according to claim 1, wherein a nematode behaviorally sensitized by a central nervous system stimulant or another dopamine agonist is used.

5. The screening method according to claim 4, wherein a nematode behaviorally sensitized by any of a psychostimulant, an apomorphine, and a narcotic analgesic is used.

6. The screening method according to claim 5, wherein a nematode behaviorally sensitized by any of amphetamine, methamphetamine, apomorphine, and cocaine is used.

7. A behaviorally sensitized nematode, which is used for screening a gene relating to a psychiatric disorder or a substance useful for prophylactic and/or therapeutic treatment of the psychiatric disorder.

8. The behaviorally sensitized nematode according to claim 7, wherein the psychiatric disorder is schizophrenia.

9. The behaviorally sensitized nematode according to claim 7, which is behaviorally sensitized by a central nervous system stimulant or another dopamine agonist.

10. The behaviorally sensitized nematode according to claim 9, which is behaviorally sensitized by any of a psychostimulant, an apomorphine, and a narcotic analgesic.

11. The behaviorally sensitized nematode according to claim 10, which is behaviorally sensitized by any of amphetamine, methamphetamine, apomorphine, and cocaine.

12. The screening method according to claim 2, wherein the psychiatric disorder is schizophrenia.

13. The screening method according to claim 2, wherein a nematode behaviorally sensitized by a central nervous system stimulant or another dopamine agonist is used.

14. The screening method according to claim 3, wherein a nematode behaviorally sensitized by a central nervous system stimulant or another dopamine agonist is used.

15. The screening method according to claim 13, wherein a nematode behaviorally sensitized by any of a psychostimulant, an apomorphine, and a narcotic analgesic is used.

16. The screening method according to claim 15, wherein a nematode sensitized by any of amphetamine, methamphetamine, apomorphine, and cocaine is used.

17. The behaviorally sensitized nematode according to claim 8, which is behaviorally sensitized by a central nervous stimulant or another dopamine agonist.

18. The behaviorally sensitized nematode according to claim 17, which is behaviorally sensitized by any of a psychostimulant, an apomorphine, and a narcotic analgesic.

19. The behaviorally sensitized nematode according to claim 18, which is behaviorally sensitized by any of amphetamine, methamphetamine, apomorphine, and cocaine.
